(19) United States
(12) Patent Application Publication
(10) Pub. No.: US 2025/0031960 A1
(43) Pub. Date: Jan. 30, 2025

(54) SYSTEM FOR OPTICAL INTERROGATION OF AN INTRAOCULAR IMPLANT THROUGH INTERFERENCE PATTERN PROJECTION

(71) Applicant: TWENTY TWENTY THERAPEUTICS LLC, South San Francisco, CA (US)

(72) Inventors: Supriyo Sinha, Menlo Park, CA (US); Oleg Rumyantsev, St. Johns, FL (US)

(21) Appl. No.: 18/781,767

(22) Filed: Jul. 23, 2024

Related U.S. Application Data
(60) Provisional application No. 63/516,485, filed on Jul. 28, 2023.

(51) Int. Cl. A61B 3/16; A61B 3/15

(52) U.S. Cl. CPC A61B 3/16; A61B 3/152

(57) ABSTRACT

An intraocular pressure (IOP) measurement system comprising: an optical pressure sensor implantable in an eye, wherein the sensor has a substrate coupled to a membrane that changes shape as a function of an intraocular pressure of the eye, and the substrate and the membrane define a sealed cavity; an optical transmitter to emit an incident optical beam to the sensor; a receiver to produce an interference pattern in response to receiving a plurality of reflections of the incident optical beam from the sensor; an image sensor operable to receive a projection of the interference pattern; and a processor configured to estimate the intraocular pressure of the eye based on processing the projection of the interference pattern.

### RELATED APPLICATION

This patent application claims the benefit of priority of U.S. Provisional Application No. 63/516,485 filed Jul. 28, 2023, which is herein incorporated by reference.

### FIELD

The subject matter of this disclosure relates to techniques for measuring an intraocular implant, and more specifically optical interrogation of a flexible membrane through interference pattern projection. Such techniques are useful for treating and/or monitoring progression of eye diseases including glaucoma, but are not limited to use with the treatment of eye disease.

### BACKGROUND

Typically, intraocular parameters within an eye, for example intraocular pressure (IOP), are measured using a tonometer. A tonometer is a device that is outside the eye and thus does not require a sensor within the eye. Contact tonometry is performed in a clinical setting, and the procedure requires numbing of the patient's eye, resulting in both inconvenience and discomfort. Noncontact tonometry involves directing a puff or jet of air towards the patient's eye and measuring the resulting deflection dynamics of the cornea. However, this requires a bulky pump arrangement that may not be practical for home use, and is not as accurate as contact tonometry. In addition, in some instances, it is desirable to be able to measure such parameters frequently and from the convenience of ones home. For example, in the case of a condition such as glaucoma, which causes chronic heightened IOP, treatment mainly involves periodically administering pharmaceutical agents to the eye to decrease IOP. These drugs can be delivered, for example, by injection or eye drops. However, effective treatment of glaucoma requires adherence to dosage schedules and a knowledge of the patient's IOP. The IOP for a given patient can vary significantly based on time of day, exercise, how recently a medication was taken, and other factors. Typically, IOP measurements are performed in a doctor's office and often no more than once or twice per year. These infrequent measurements are less able to account for variation in the patient's IOP, and may become stale due to the length of time between them. This means that any given measurement is subject to uncertainty, so it may take several IOP measurements over time to have confidence in the health of the patient's eye.

A wireless, implantable, continuous IOP monitoring system has been suggested that has a commercial pressure sensing element with digital readout, and a microelectronic chip that supports wireless power/data telemetry and a wired serial communication interface with the pressure sensing element. An on-chip integrated RF coil receives power from near-field RF coupling at 915 MHz, and transmits pressure measurement bits via RF-backscattering to an external reader. To ensure accurate measurements by the external reader, however, it is critical that the external reader be precisely aligned with the pressure sensing element.

### SUMMARY

An aspect of the disclosure is a system and process for measurement of an intraocular parameter from a miniature implant (e.g., about 0.2-0.5 mm thick in the z direction and/or about 1 mm diameter) using interference pattern projection. In some aspects, the intraocular parameter being measured may be the IOP of the eye. Representatively, in some aspects, a reader or sensor reading device (an electronic device that may be portable, battery powered, and held by the person themselves) is aligned with the eye, and an optical beam is emitted by a transmitter inside the reader. The reader is not physically attached to the sensor and may be outside of the eye. The beam may travel through free space (the ambient environment outside of the eye) and then enters the cornea where it impinges upon the sensor and is reflected by the sensor, towards a receiver in the reader. The reflection changes as it follows the changing IOP of the eye (for example over the course of a day). The sensor may be passive in that it does not have a source of stored power that is used to transmit a signal containing information about the IOP. Instead, the sensor may include a rigid surface and a flexible membrane that changes shape as a result of the changes in eye pressure acting on the membrane. The incident beam on this membrane will bend or compress, as a function of the nearby IOP, resulting in the reflection changing accordingly. An estimate of the IOP is then determined by digitally processing an electrical output signal of the receiver (that is responsive to the reflections that travelled from the sensor and then through the ambient environment before impinging on the receiver.)

Various interferometric techniques have been previously proposed to interrogate such sensors with a beam of light in order to get a reading of the flexible membrane's deflection and pressure acting on the membrane using a handheld device as previously discussed. The major issue in such arrangement is the need for precise spatial alignment between the sensor and the handheld device. Since both the user's hands and eyes are moving, performing such measurement easily and reliably is problematic. In order to decrease alignment sensitivity, several attempts have been made to design or alter both the handheld device and the implant. One such technique includes the use of an “eyebox” volume in which the sensor needs to be positioned with respect to the handheld device for a proper reading. The eyebox may be, for example, at least a few hundred microns per side and defined as an area or a volume within which the transmitter, receiver and sensor should be located in order to produce reliable measurements. This might still be problematic to achieve, especially for people with age related tremors, or other conditions making it difficult for them to position the handheld device within such a small volume. The instant disclosure proposes an alternative interferometric technique for optical interrogation of an IOP sensor implant. This approach allows for an increase in the eyebox volume by a factor of ˜10-100. Representatively, the eyebox volume may be enlarged to a size ˜10-100 times greater than approximately 2 mm×2 mm×0.2 mm, for example a volume size of up to approximately 3 mm×4 mm×5 mm or more. This, in turn, makes alignment of the handheld device with the implant and measurement significantly easier for the user. To achieve this, the system uses a projection of the interferometric pattern or profile of the interfering reflections from the sensor. Use of the projection eliminates the need for precise special alignment between the sensor and the device therefore allowing for reading and/or measurement of the sensor within a larger eyebox volume.

Representatively, in one aspect, an intraocular pressure (IOP) measurement system includes an optical pressure sensor implantable in an eye, wherein the sensor has a substrate coupled to a membrane that changes shape as a function of an intraocular pressure of the eye, and the substrate and the membrane define a sealed cavity; an optical transmitter to emit an incident optical beam to the sensor; a receiver to produce an interference pattern in response to receiving a plurality of reflections of the incident optical beam from the sensor; an image sensor operable to receive a projection of the interference pattern; and a processor configured to estimate the intraocular pressure of the eye based on processing the projection of the interference pattern. In some aspects, the plurality of reflections are frequency dependent reflections of the incident optical beam that change as a function of the intraocular pressure to produce the interference pattern. In still further aspects, the plurality of reflections include a first reflection from the interface between the membrane and the cavity, and a second reflection from the interface between the substrate and the cavity. In another aspect, the interference pattern includes a number of concentric rings and the number of concentric rings is a function of a shape of the membrane. The interference pattern may include a number of concentric rings and each ring of the number of concentric rings may include a position that is a function of the shape of the membrane. In some aspects, the processor determines, by processing the projection of the interference pattern, whether the projection of the interference pattern matches a theoretical interference profile to estimate the intraocular pressure. In some aspects, the theoretical interference profile corresponds to a known membrane pressure. In some aspects, the transmitter and the receiver are integrated within a reader and the reader is operable to read the optical pressure sensor when the reader and the sensor are positioned within a volume between 10 to 100 times greater than a volume of 2 mm×2 mm×0.2 mm. The transmitter, the receiver and the image sensor may be integrated within a single housing of a handheld device. The sensor may be implanted in a cornea of the eye.

In another aspects, a method for measuring intraocular pressure of an eye includes emitting an optical beam toward the eye; detecting, as an output signal, an interference pattern corresponding to a plurality of reflections of the optical beam from a pressure sensor that is implanted in the eye; projecting the interference pattern to an image sensor; and processing the projected interference pattern to compute an estimate of the intraocular pressure of the eye. In some aspects, processing the output signal includes performing an interferometric image processing algorithm. In some aspects, the interferometric image processing algorithm analyzes the projected interference pattern and matches the projected interference pattern to a theoretical interference pattern that corresponds to a known pressure on the membrane to determine the intraocular pressure of the eye. The sensor may include a rigid substrate and a flexible membrane attached to the substrate that define a sealed cavity. The plurality of reflections may be frequency dependent reflections of the incident optical beam that change as a function of the intraocular pressure to produce the interference pattern. The plurality of reflections may include a first reflection from the interface between the membrane and the cavity, and a second reflection from the interface between the substrate and the cavity. The emitting and the detecting may be performed by a transmitter and a receiver, and the transmitter, the receiver and the pressure sensor are positioned within a volume of between 10 to 100 times greater than a volume of 2 mm×2 mm×0.2 mm during measuring. The transmitter, the receiver and the image sensor may be integrated within a single housing of a handheld device. In some aspects, a projector integrated within the single housing of the handheld device may project the interference pattern to the image sensor. In some aspects, the sensor may be implanted in a cornea of the eye.

The above summary does not include an exhaustive list of all aspects of the present disclosure. It is contemplated that the disclosure includes all systems and methods that can be practiced from all suitable combinations of the various aspects summarized above, as well as those disclosed in the Detailed Description below and particularly pointed out in the Claims section. Such combinations may have particular advantages not specifically recited in the above summary.

### BRIEF DESCRIPTION OF THE DRAWINGS

Several aspects of the disclosure here are illustrated by way of example and not by way of limitation in the figures of the accompanying drawings, in which like references indicate similar elements. It should be noted that references to “an” or “one” aspect in this disclosure are not necessarily to the same aspect, and they mean at least one. Also, in the interest of conciseness and reducing the total number of figures, a given figure may be used to illustrate the features of more than one aspect of the disclosure, and not all elements in the figure may be required for a given aspect.

FIG. 1 shows a schematic diagram of an example system including a sensor and a reader device for measuring an intraocular parameter of an eye.

FIG. 2 shows a schematic diagram of a user holding a reader device close to their eye for measuring an intraocular parameter of the eye of FIG. 1.

FIG. 3 shows a schematic diagram of an example system for measuring an intraocular parameter of the eye.

FIG. 4 shows a schematic diagram of an example sensor for measuring an intraocular parameter.

FIG. 5 shows a block diagram of an exemplary process for measuring an intraocular parameter.

### DETAILED DESCRIPTION

Several aspects of the disclosure with reference to the appended drawings are now explained. Whenever the shapes, relative positions and other aspects of the parts described are not explicitly defined, the scope of the invention is not limited only to the parts shown, which are meant merely for the purpose of illustration. Also, while numerous details are set forth, it is understood that some aspects of the disclosure may be practiced without these details. In other instances, well-known circuits, structures, and techniques have not been shown in detail so as not to obscure the understanding of this description.

The terminology used herein is for the purpose of describing particular aspects only and is not intended to be limiting of the disclosure. Spatially relative terms, such as “beneath”, “below”, “lower”, “above”, “upper”, and the like may be used herein for ease of description to describe one element's or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (e.g., rotated 90 degrees or at other orientations) and the spatially relative descriptors used herein interpreted accordingly.

As used herein, the singular forms “a”, “an”, and “the” are intended to include the plural forms as well, unless the context indicates otherwise. It will be further understood that the terms “comprises” and/or “comprising” specify the presence of stated features, steps, operations, elements, and/or components, but do not preclude the presence or addition of one or more other features, steps, operations, elements, components, and/or groups thereof.

The terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

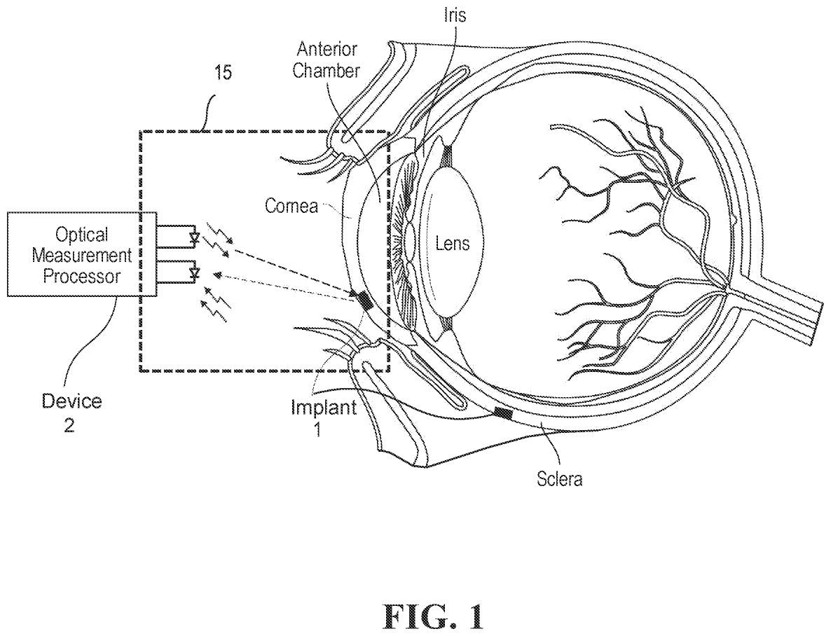

FIG. 1 shows an example system for measuring an intraocular parameter, using a handheld device 2 (e.g., a reader device) and an implant 1 (e.g., an IOP sensor). FIG. 1 shows the implant (including a sensor) implanted in its entirety within the cornea, or alternatively in its entirety within the sclera. While the drawings in this disclosure may not be to scale, they do illustrate that the sensor or implant 1 is small enough to be implanted into a typical cornea or sclera. Sensor 1 may be a passive sensor in that it does not have a source of stored power that is used to transmit a signal containing information about the IOP. Instead, a part of sensor 1 is designed to bend or compress or conform according to the IOP, and that part is also designed to reflect incident optical energy. As a result, sensor or implant 1 changes how it reflects the optical energy, as a function of the IOP at that moment. Thus, the reflection changes in that it follows the changing IOP (in the anterior chamber), for example over the course of a day. As the sensor is passive, it can be made small and thin so as to be implanted into the cornea in a minimally invasive manner, more easily and with less risk of complications as compared to implant locations that are further inside the eye. The implant can be made sufficiently small, e.g., having a footprint or area in the x-y plane of about 1 mm² (or a diameter of about 1 mm) and a thickness in the z direction of about 0.2 to 0.5 mm, so that it is entirely embedded in the cornea or sclera as shown.

Device 2 may include a transmitter and a receiver. The transmitter may be a single photo-emitter or monochromatic point light source, or it may be an array of light emitters. In some aspects, the emitted beam may be directional, having a narrow or directional primary lobe aimed at sensor or implant 1. In other aspects, the emitted beam may be a collimated beam having multiple parallel light rays. The receiver may be a single photo-detector, or it may be a one-dimensional or two-dimensional array of photo-detectors (the latter being especially useful for the case where the processor is determining the interference pattern via for example an imaging function being performed upon the signals produced by the array of photo-detectors.) The transmitter and receiver of device 2 must be properly positioned relative to sensor or implant 1 to ensure an accurate measurement. For example, device 2 and implant or sensor 1 may be positioned within an eyebox volume 15 as illustrated by the dashed line to ensure an accurate measurement. The IOP is then measured by detecting changes in the cornea that cause the implanted sensor to bend or compress, during reflection of the incident beam that is detected by the receiver.

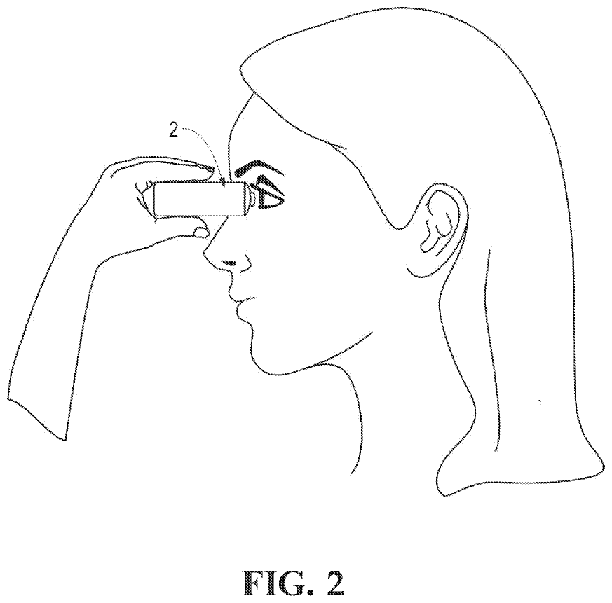

FIG. 2 illustrates a user holding the device 2 in their hand, and who has brought the handheld device close to their eye to read sensor or implant 1. In this aspect, the handheld device 2 is a portable or handheld device configured to read the measured intraocular data once the handheld device 2 is close to the eye and aligned with sensor 1. For example, handheld device 2 may be deemed to be in a proper position and aligned with sensor or implant 1 when device 2 determines that they are both within an eyebox volume (e.g., volume 15) as previously discussed. Once device 2 determines a proper position is achieved, device 2 can then read the measured pressure data from sensor or implant 1 and inform the user about the measured intraocular parameter.

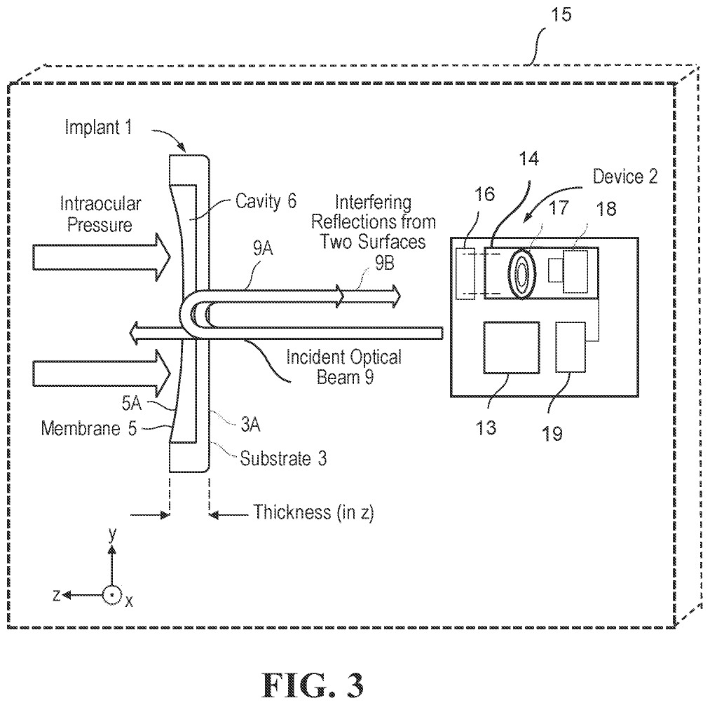

Representatively, FIG. 3 illustrates a general overview of sensor or implant 1 and device 2. Sensor or implant 1 may include a rigid substrate 3 to which a flexible membrane 5 (the terms rigid and flexible being relative to one another) is attached, so as to form a sealed cavity 6. Sealed cavity 6 may be bound by (or defined by) the internal faces of substrate 3 and membrane 5. Membrane 5 may be spaced from a rigid surface 3A of substrate 3 in the thickness or z-direction for example between 5 to 30 microns. Membrane 5 may be made of or coated with one or more of the following biocompatible materials: silica or any material that has a suitable difference in optical index of refraction relative to that of the surrounding tissue. Substrate 3 may be made or coated with any one of similar biocompatible materials as well. The indices of refraction of the membrane and substrate surface should be chosen to maximize the contrast of the interference that is due to reflections from those surfaces of the sensor or implant 1. Sealed cavity 6 may be a gaseous volume that is made to be at a low enough pressure, for example on the order of the atmosphere (atm) or lower, that allows changes in the IOP to sufficiently bend or change the shape of the membrane 5 (via movement of the corneal tissue that surrounds sensor 1 and that is caused by the changing IOP) so as to be detectable in the reflections. When sensor or implant 1 is implanted in the orientation shown, namely that an outside surface of the membrane 5 faces the inside of the eye (or faces the anterior chamber of the eye) while the outside surface of the substrate 3 faces the transmitter 13 (or faces the outside environment of the eye), the incident beam 9 will be reflected as at least two interfering reflections 9A, 9B shown.

To make IOP measurements, reader or device 2 contains optical transmitter 13 and optical receiver 14, both of which may be integrated within a housing of the reader or device 2. Reader or device 2 is aligned with the eye (as shown in FIGS. 1-2) so that optical beam 9 of incident optical energy (radiation or waves) emitted by the transmitter 13 impinges on sensor 1, while reflections 9A, 9B of that radiation from the sensor or implant 1 are detected by the receiver 14 (as its output signal.) Note the term “beam” is used generically here, and does not require a beam forming transmitter array. Rather transmitter 13 may be (or include) any kind of light source that emits a beam 9. For example, transmitter 13 may include a laser diode (LD) that emits a monochromatic (e.g., approximately 750 nm wavelength) point light source. Beam 9 enters the cornea where it impinges (or is incident) upon sensor or implant 1, and is reflected by surfaces of the sensor 1 towards the receiver.

Representatively, in the particular example of FIG. 3, sensor 1 is oriented so that the incident beam 9 impinges upon sensor or implant 1 and creates two interfering reflections 9A, 9B. For example, reflection 9A may be a reflection off the interface between membrane surface 5A and cavity 6, and reflection 9B may be a reflection from the interface between rigid surface 3A of substrate 3 and cavity 6. Reflections 9A, 9B may therefore be considered frequency dependent reflections of the incident optical beam 9 that may change depending on the shape of membrane 5 which, in turn, is a function of the pressure in the eye. The corresponding interference reflection pattern or profile of reflections 9A, 9B will therefore also change as a function of the pressure on the membrane and may therefore be used to determine the pressure in the eye. It is noted that while other reflections are possible, they are minimal due to low difference in refractive index between the aqueous humor in the eye and membrane/substrate materials. The reflections and/or the corresponding interference profiles may be received by the receiver 14 and an estimate of IOP is then determined based on the received reflections/interference profiles.

As previously discussed, to ensure an accurate reading and measurements, device 2 must be properly positioned relative to sensor or implant 1. For example, device 2 and sensor or implant 1 may be positioned within eyebox 15 (e.g., an area or a volume within which the transmitter, receiver and sensor should be located in order to produce reliable measurements) as illustrated by the dashed lines. Conventional eyebox volumes, however, have typically been relatively small. Moreover, once within the eyebox, a precise angular alignment between an optical axis (e.g., the incident beam emitted by the transmitter) of device and sensor or implant was often required to ensure that the reflected beams are received by the device. Such tight tolerances, however, may be difficult to meet for users with age related conditions (e.g., tremors), or other conditions making it difficult for them to position the handheld device so precisely relative to the implant or sensor. To alleviate these challenges, system disclosed herein may further implement an interferometric technique which does not require focusing of the interfering reflections for optical interrogation of the sensor or implant. Instead, a projection of an interference pattern of the reflections is used for optical interrogation of the implant or sensor 1. Using this improved interferometric technique (eliminating focusing) allows for an increase in the eyebox volume 15 (as compared to a system not implementing the disclosed technique) by a factor of ˜10-100. Representatively, the eyebox volume may be enlarged to a size ˜10-100 times greater than approximately 2 mm×2 mm×0.2 mm, for example a volume size of up to approximately 3 mm×4 mm×5 mm or more. In addition, the system is relatively insensitive to any angular misalignment between sensor 1 and device 2 in dimensions normal to implant or sensor 1. In other words, once device 2 and implant or sensor 1 are determined to be within eyebox 15, measuring may proceed without meeting additional angular alignment or positioning parameters.

Representatively, the system may further include an interference pattern projector 16 that is configured to project an interference pattern or profile 17 of interfering reflections 9A, 9B onto image sensor 18. In some aspects, projector 16 may be integrated into device 2 as part of receiver 14. For example, receiver 14 may receive interfering reflections 9A, 9B from implant or sensor 1 and produce interference pattern or profile 17 based on the reflections. Pattern or profile 17 may then be communicated to projector 16 which then projects pattern or profile 17 onto image sensor 18. In some aspects, image sensor 18 may be part of a camera integrated into device 2.

Representatively, during operation, transmitter 13 may emit a monochromatic (λ≈750 nm) point light source such as a laser diode (LD) coupled to a single-mode optical fiber that illuminates implant or sensor 1 from a distance of approximately 15-20 mm. The cone of light that hits implant or sensor 1 may create two reflections 9A, 9B as previously discussed. The two reflections 9A, 9B interfere differently (e.g. constructively/destructively) at different positions on the membrane, depending on the local thickness of the cavity 6. This results in an interference profile 17 that is a function of the shape of the membrane. The interference profile 17 then gets projected by projector 16 onto image sensor 18. A processor 19 that communicates with image sensor 18 may include a computer image processing algorithm that analyzes the recorded interference pattern or profile 17 and matches it to either calibration data or theoretical interference profiles to determine the pressure acting on the membrane 5. In addition, in some aspects, to compensate for the positive optical power of the cornea, a lens (such as matched aspheric lens pair) may relay the point source closer to the eye.

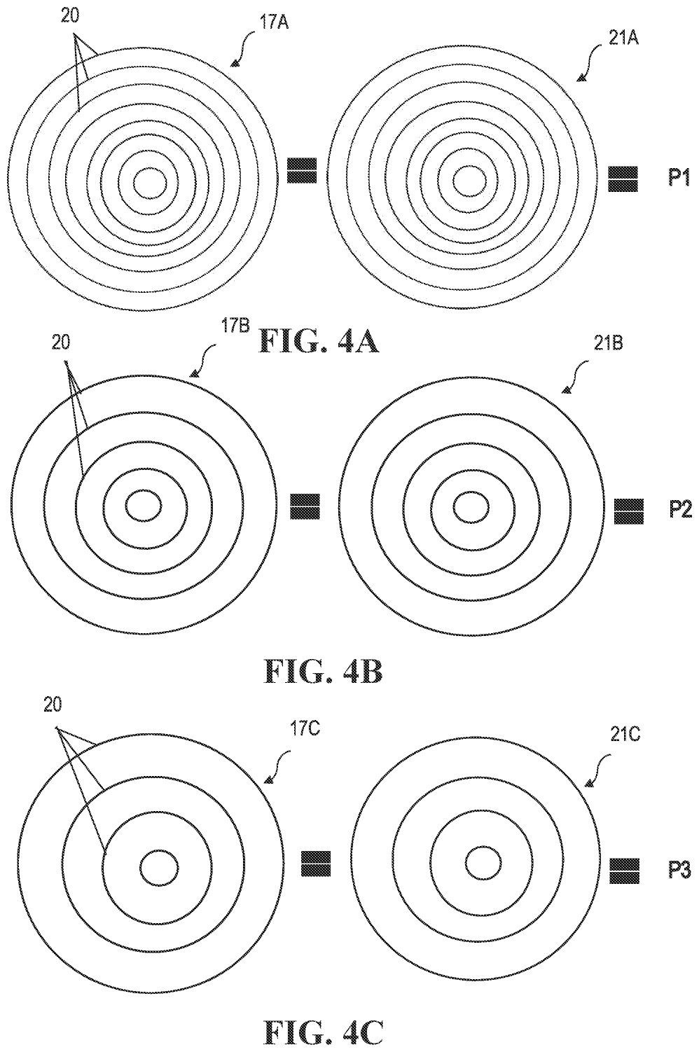

Representatively, FIGS. 4A, 4B and 4C illustrate a number of exemplary interference profiles that may be projected onto image sensor 18 and matched to either calibration data or theoretical interference profiles to determine the pressure. It should be understood that for a bent circular membrane (e.g., membrane 5), the interference profiles 17A, 17B, 17C will appear as concentric interference rings 20 as shown in FIG. 4A-C. It is contemplated, however, that the technique disclosed herein may apply to other patterns and profiles resulting from membranes having different shapes and sizes. The outer diameter of the pattern 17A-C corresponds to the known diameter of the implant (without interference, reflections from the implant would look like a bright disk of light with sharp edges). The number and positions of the interference rings 20 are a function of the shape of the membrane (e.g., membrane 5). In this aspect, the number and positions of the interference rings 20, or more generally the whole two-dimensional interference profile, can be fitted with a theoretical profile that depends on the shape of the membrane, which in turn depends on the pressure. Resolution of such a projection system (i.e. the number of resolvable fringes) is set by the diffraction limit. Representatively, for illumination with λ=750 nm light, implant diameter d=1 mm and distance from sensor to implant L=20 mm, the minimum resolvable distance may be determined by the following formula:

$$Δ = λ / d * L \approx 15 um$$

In other words, approximately thirty (30) interference rings 20 (“fringes”) can be resolved by this method, and in turn, a pressure acting on membrane 5 may be determined. For example, interference pattern or profile 17A may match a theoretical interference profile 21A that corresponds to a known or predetermined pressure P1. Interference pattern or profile 17B may match a theoretical interference profile 21B that corresponds to a known or predetermined pressure P2. Interference pattern or profile 17C may match a theoretical interference profile 21C that corresponds to a known or predetermined pressure P3. Based on the pressure acting on membrane 5 (e.g., P1, P2, P3), an estimate of the IOP may be determined. In some aspects, to determine the pressure in the eye relative to the ambient pressure (which is typically what is needed for IOP), an ambient pressure sensor could also be used in the reader and this reading can be subtracted from the absolute pressure reading. In still further aspects, the reader may also include an ambient temperature monitor to calibrate for any temperature dependent performance of the implant. In addition, device 2 may include accelerometers to determine the orientation of the implant.

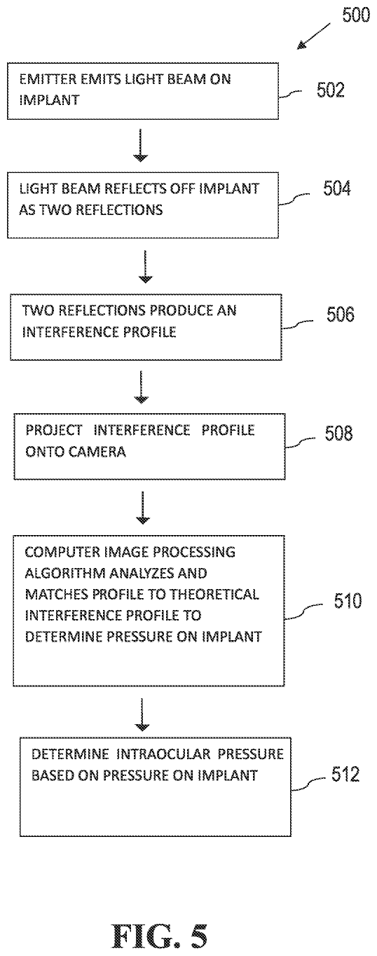

Referring now to FIG. 5, FIG. 5 illustrates one representative IOP measurement process 500 implementing the interference pattern projecting technique disclosed herein. Representatively, process 500 may include emitting a light beam (e.g., beam 9) onto the implant or sensor (e.g., implant or sensor 1) at operation 502. The light beam may then be reflected off of the implant or sensor as a number of reflections, for example two reflections (e.g., reflections 9A, 9B) at operation 504. The two reflections produce an interference pattern or profile at operation 506, which is projected onto a camera at operation 508. At operation 510, a computer image processing algorithm analyzes and matches the pattern to a theoretical interference profile to determine a pressure on the membrane (e.g., membrane 5). Based on the pressure on the membrane, the IOP is then determined at operation 512.

The interference projecting technique disclosed herein has multiple advantages over other interferometric techniques. For example, the projection technique increases the eyebox volume significantly and decreases misalignment sensitivities. The technique is by design almost insensitive to misalignment in dimension normal to the implant. In contrast, other techniques for optical interrogation require positioning of an implant in the focus of an optical system, which in turn can lead to high sensitivity to alignment and poor usability. For example, as previously discussed, the projection system disclosed herein allows for a total volume in which the implant can be put with respect to the implant in order to get a measurement on the order of ˜3 mm×4 mm×5 mm, which is ˜100 times larger (in volume) compared to other techniques due to elimination of focusing. In addition, due to the small number of components, the proposed handheld device is simple, and maintenance-free.

While certain aspects have been described and shown in the accompanying drawings, it is to be understood that such are merely illustrative of and not restrictive on the broad invention, and that the invention is not limited to the specific constructions and arrangements shown and described, since various other modifications may occur to those of ordinary skill in the art. For example, it should be understood that while a passive implant or sensor is primarily disclosed herein, in some aspects, the sensor or implant may be considered an active sensor and have a source of stored power that is used to transmit a signal containing information about the IOP. Moreover, although a single mode fiber coupled light source is disclosed, a vertical-cavity surface-emitting laser (VCSEL) or an edge emitting source with appropriate collimation optics could be used. Still further, while projector is described as integrated into a receiver component of the handheld device, in some aspects, projector may be integrated into the implant or sensor and project the interference pattern from the implant or sensor to the handheld device to then be received and processed by the handheld device. In addition, the aspects disclosed herein are not limited to ophthalmological/medical sensing applications and can be used in a much broader range of measurement applications involving repetitive repositioning of the miniature sensor and an external optical measurement device. The description is thus to be regarded as illustrative instead of limiting.

What is claimed is:

1. An intraocular pressure (IOP) measurement system comprising:
an optical pressure sensor implantable in an eye, wherein the sensor has a substrate coupled to a membrane that changes shape as a function of an intraocular pressure of the eye, and the substrate and the membrane define a sealed cavity;
an optical transmitter to emit an incident optical beam to the sensor;
a receiver to produce an interference pattern in response to receiving a plurality of reflections of the incident optical beam from the sensor;
an image sensor operable to receive a projection of the interference pattern; and
a processor configured to estimate the intraocular pressure of the eye based on processing the projection of the interference pattern.

2. The system of claim 1 wherein the plurality of reflections are frequency dependent reflections of the incident optical beam that change as a function of the intraocular pressure to produce the interference pattern.

3. The system of claim 1 wherein the plurality of reflections comprises a first reflection from an interface between the membrane and the cavity, and a second reflection from the interface between the substrate and the cavity.

4. The system of claim 1 wherein the interference pattern comprises a number of concentric rings and the number of concentric rings is a function of a shape of the membrane.

5. The system of claim 1 wherein the interference pattern comprises a number of concentric rings and each ring of the number of concentric rings comprises a position that is a function of the shape of the membrane.

6. The system of claim 1 wherein the processor determines, by processing the projection of the interference pattern, whether the projection of the interference pattern matches a theoretical interference profile to estimate the intraocular pressure.

7. The system of claim 6 wherein the theoretical interference profile corresponds to a known membrane pressure.

8. The system of claim 1 wherein the transmitter and the receiver are integrated within a reader and the reader is operable to read the optical pressure sensor when the reader and the sensor are positioned within a volume between 10 to 100 times greater than a volume of 2 mm×2 mm×0.2 mm.

9. The system of claim 1 wherein the transmitter, the receiver and the image sensor are integrated within a single housing of a handheld device.

10. They system of claim 1 wherein the sensor is implanted in a cornea of the eye.

11. A method for measuring intraocular pressure of an eye, the method comprising:
emitting an optical beam toward the eye;
detecting, as an output signal, an interference pattern corresponding to a plurality of reflections of the optical beam from a pressure sensor that is implanted in the eye;
projecting the interference pattern to an image sensor; and
processing the projected interference pattern to compute an estimate of the intraocular pressure of the eye.

12. The method of claim 11 wherein processing the output signal comprises performing an interferometric image processing algorithm.

13. The method of claim 12 wherein the interferometric image processing algorithm analyzes the projected interference pattern and matches the projected interference pattern to a theoretical interference pattern that corresponds to a known pressure to determine the intraocular pressure of the eye.

14. The method of claim 11 wherein the sensor comprises a rigid substrate and a flexible membrane attached to the substrate that define a sealed cavity.

15. The method of claim 14 wherein the plurality of reflections are frequency dependent reflections of an incident optical beam that change as a function of the intraocular pressure to produce the interference pattern.

16. The method of claim 14 wherein the plurality of reflections comprises a first reflection from an interface between the flexible membrane and the cavity, and a second reflection from the interface between the substrate and the cavity.

17. The method of claim 11 wherein the emitting and the detecting are performed by a transmitter and a receiver, and the transmitter, the receiver and the pressure sensor are positioned within a volume of between 10 to 100 times greater than a volume of 2 mm×2 mm×0.2 mm during measuring.

18. The method of claim 17 wherein the transmitter, the receiver and the image sensor are integrated within a single housing of a handheld device.

19. The method of claim 18 further comprising a projector integrated within the single housing of the handheld device to project the interference pattern to the image sensor.

20. The method of claim 11 wherein the sensor is implanted in a cornea of the eye.
